(19) United States
(12) Patent Application Publication
(10) Pub. No.: US 2018/0271484 A1
(43) Pub. Date: Sep. 27, 2018

(54) METHOD AND SYSTEMS FOR A HAND-HELD AUTOMATED BREAST ULTRASOUND DEVICE

(71) Applicant: General Electric Company, Schenectady, NY (US)

(72) Inventor: Doug Whisler, Seattle, WA (US)

(21) Appl. No.: 15/465,510

(22) Filed: Mar. 21, 2017

(51) Int. Cl. A61B 8/00; A61B 8/08

(52) U.S. Cl. CPC A61B 8/4444; A61B 8/4483; A61B 8/5207; A61B 8/5215; A61B 2562/0261; A61B 8/467; A61B 8/461; A61B 8/0825; A61B 8/54

(57) ABSTRACT

Various methods and systems are provided for ultrasonically scanning a tissue sample using a hand-held automated ultrasound system. In one example, a system for ultrasonically scanning a tissue sample includes a hand-held ultrasound probe including a housing and a transducer module comprising a transducer array of transducer elements, one or more position sensors coupled within the housing, and a controller. The controller is configured to generate one or more images based on ultrasound data acquired by the transducer module and further based on position sensor data collected by the one or more position sensors.

### FIELD

Embodiments of the subject matter disclosed herein relate to medical imaging and the facilitation of ultrasonic tissue scanning.

### BACKGROUND

Volumetric ultrasound scanning of the breast may be used as a complementary modality for breast cancer screening. Volumetric ultrasound scanning usually involves the movement of an ultrasound transducer relative to a tissue sample and the processing of resultant ultrasound echoes to form a data volume representing at least one acoustic property of the tissue sample. Whereas a conventional two-dimensional x-ray mammogram only detects a summation of the x-ray opacity of individual slices of breast tissue over the entire breast, ultrasound can separately detect the sonographic properties of individual slices of breast tissue, and therefore may allow detection of breast lesions where x-ray mammography alone fails. Further, volumetric ultrasound offers advantages over x-ray mammography in patients with dense breast tissue (e.g., high content of firogladular tissues). Thus, the use of volumetric ultrasound scanning in conjunction with conventional x-ray mammography may increase the early breast cancer detection rate.

### BRIEF DESCRIPTION

In one embodiment, a system for ultrasonically scanning a tissue sample includes a hand-held ultrasound probe including a housing and a transducer module comprising a transducer array of transducer elements, one or more position sensors coupled within the housing, and a controller. The controller is configured to generate one or more images based on ultrasound data acquired by the transducer module and further based on position sensor data collected by the one or more position sensors.

In this way, the position sensor data may be used to generate a three-dimensional volume representation of the scanned tissue sample from the acquired ultrasound data. Then, images may be generated from the volume. In one example, the generated images may be tagged or otherwise associated with positional information based on the position sensor data. By doing so, a semi-automated, volumetric ultrasound may be performed using a hand-held ultrasound probe. The semi-automated nature of the ultrasound may enable subsequent ultrasounds to be performed that generate images in the same plane, at the same location, as the initial ultrasound. Such a configuration may allow the same tissue to be repeatably imaged in a highly accurate manner, aiding in detection of lesions or other diagnostic features.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIGS. 1-2 show schematic of various system components of a hand-held automated breast ultrasound device according to an embodiment of the invention.

FIG. 3 shows a cross-section of a transducer array of a hand-held automated breast ultrasound device according to an embodiment of the invention.

FIGS. 4A-4B illustrate a method for a hand-held automated breast ultrasound device.

FIGS. 5, 6, 8, and 9 illustrate example user interfaces that may be displayed to an operator during a semi-automated breast exam.

FIGS. 7 and 10 illustrate example sweeps of a hand-held automated breast ultrasound device during a semi-automated breast exam.

### DETAILED DESCRIPTION

The following description relates to various embodiments of a hand-held ultrasound device configured to perform automated breast ultrasound (ABUS) scanning. X-ray mammography is the most commonly used imaging method for mass breast cancer screening. However, x-ray mammograms only detect a summation of the x-ray opacity of individual slices over the entire breast. Alternatively, ultrasound imaging can separately detect sonographic properties of individual slices of breast tissue, thereby enabling users to detect breast lesions where x-ray mammography alone may fail.

Another well-known shortcoming of x-ray mammography practice is found in the case of dense-breasted women, including patients with high content of fibroglandular tissues in their breasts. Because fibroglandular tissues have higher x-ray absorption than the surrounding fatty tissues, portions of breasts with high fibroglandular tissue content are not well penetrated by x-rays and thus the resulting mammograms contain reduced information in areas where fibroglandular tissues reside. Thus, the use of volumetric ultrasound scanning in conjunction with conventional x-ray mammography may increase the early breast cancer detection rate.

In some examples, breast cancer detection may be improved by comparing same-patient breast exam images collected over time, such as images from exams taken every six months, every year, etc. Such “compare-to-prior” workflow practices may be aided by an automated breast ultrasound scanning device. Typical ABUS devices may include a relatively large transducer array that is automatically swept along a single axis (e.g., along a vertical axis), in order to capture an ultrasound data volume without requiring an operator to reposition the ABUS device along additional axes (e.g., the horizontal axis). However, such a configuration requires the ABUS device to be large and expensive, limiting the use of the ABUS device. Further, while these ABUS devices may be sized to capture an entirety of the breast in a single sweep, nearby tissue, such as the tissue along the chest wall under an arm, may be missed, leading to undetected lesions in some examples.

Thus, to reduce costs of the volumetric ultrasound scanning apparatus while also expanding the tissue area that may be imaged, it may be desirable to package the transducer of the volumetric ultrasound scanning apparatus in a compact, hand-held housing. Given the size of the hand-held ultrasound transducer, multiple parallel sweeps of subject tissue may be required to adequately image the breast, under arm, and other areas. However, such multiple sweeps may make ultrasound data registration onto a common volume difficult, due to operator uncertainty in positioning the transducer between sweeps, leading to inaccuracies of images taken from the ultrasound data volume.

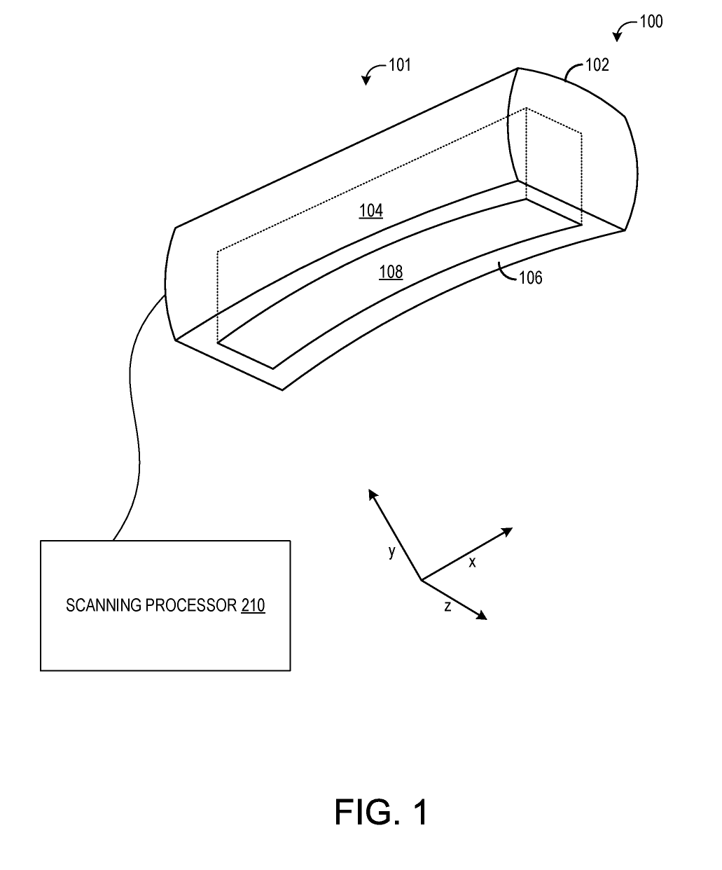

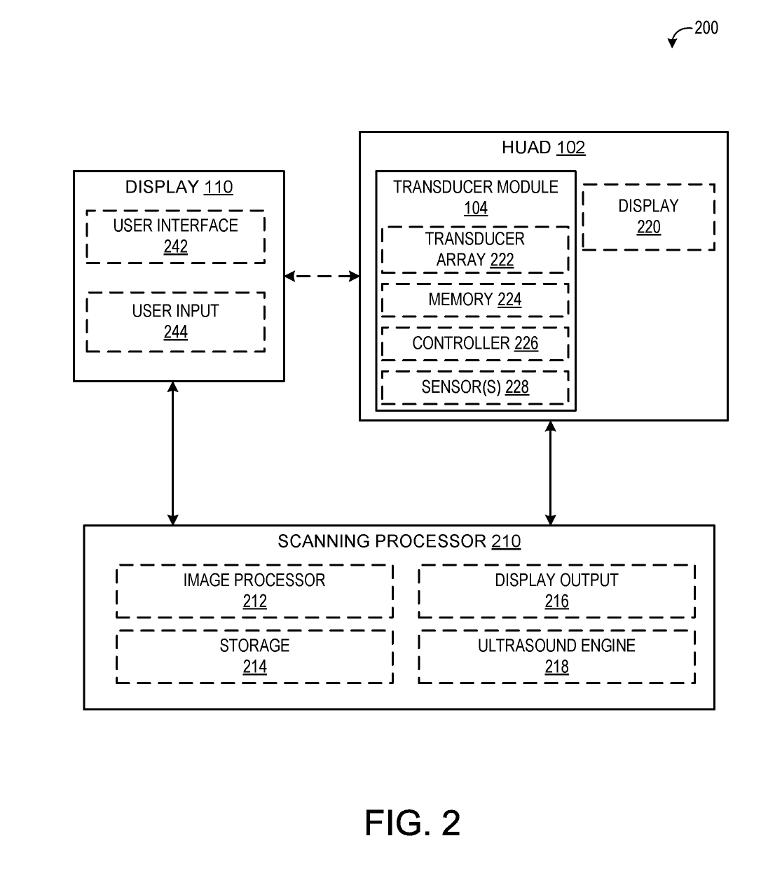

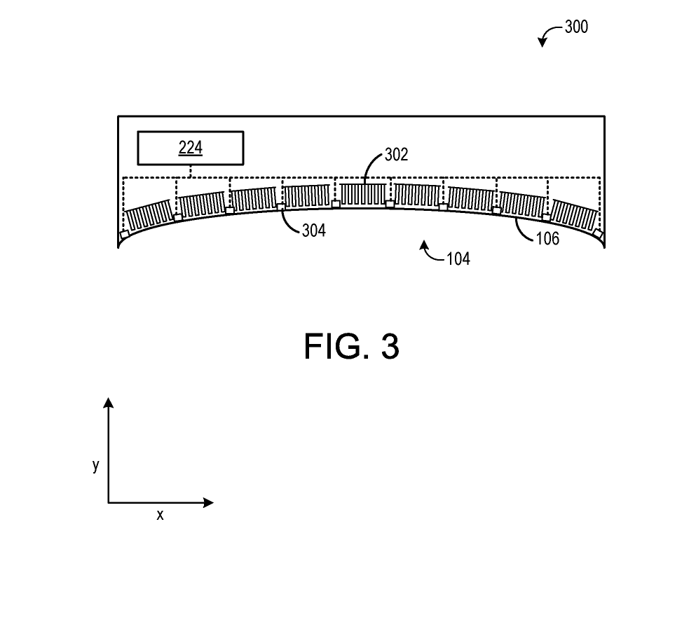

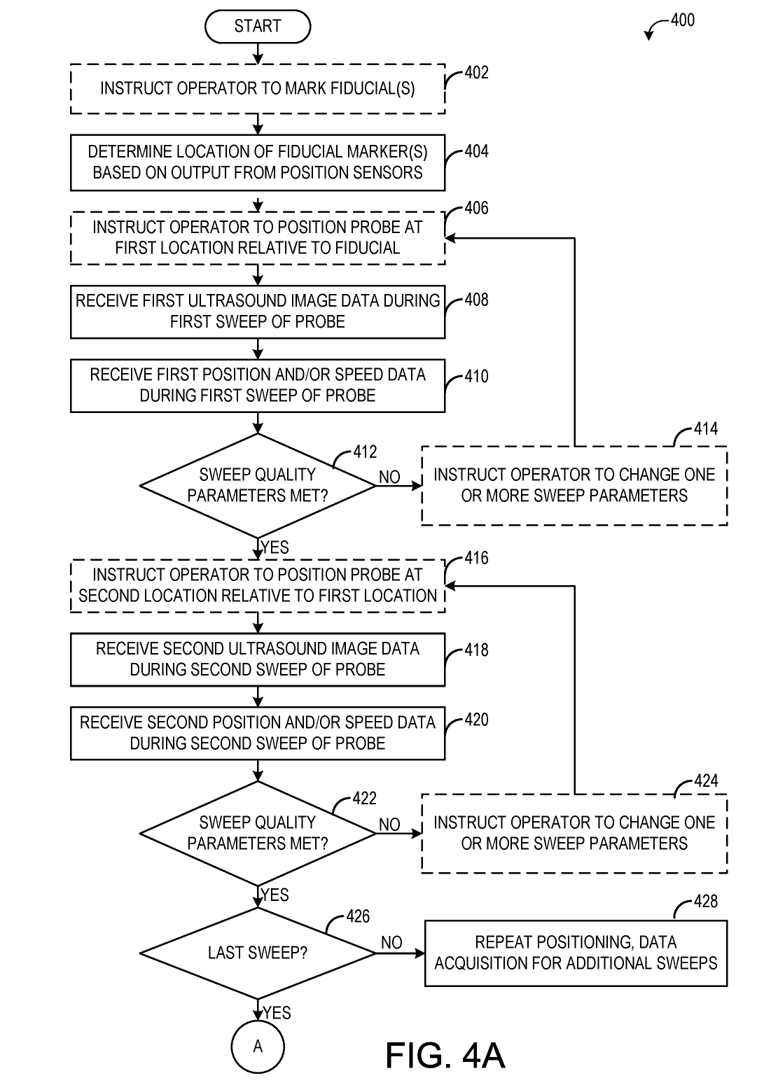

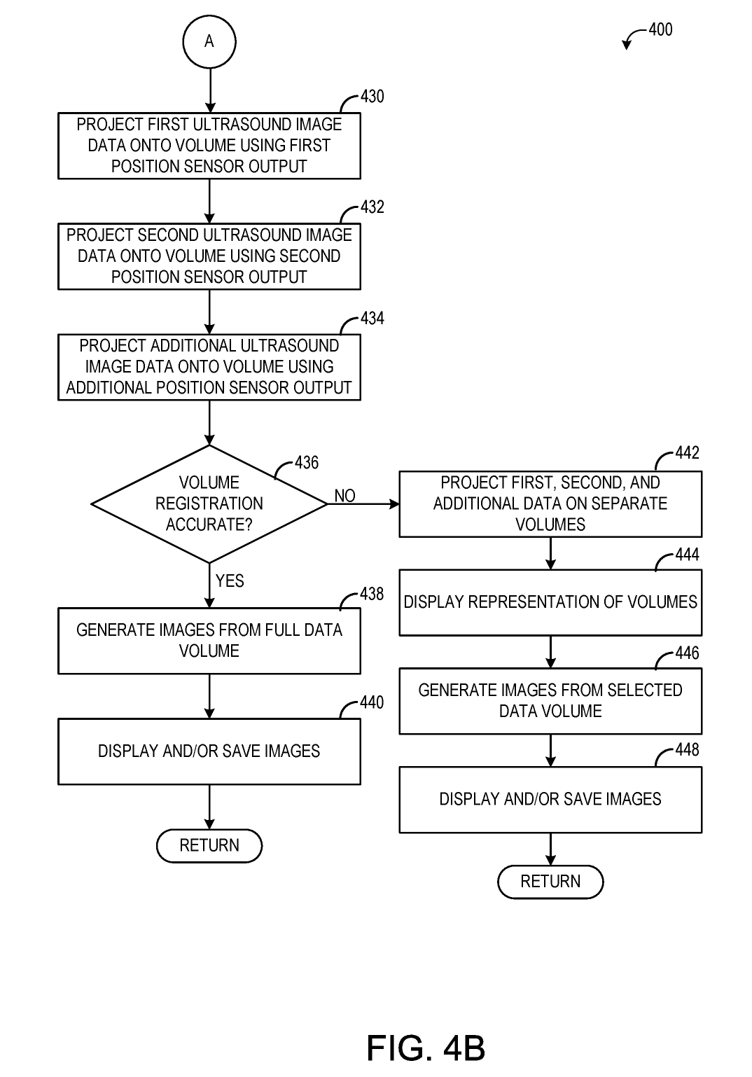

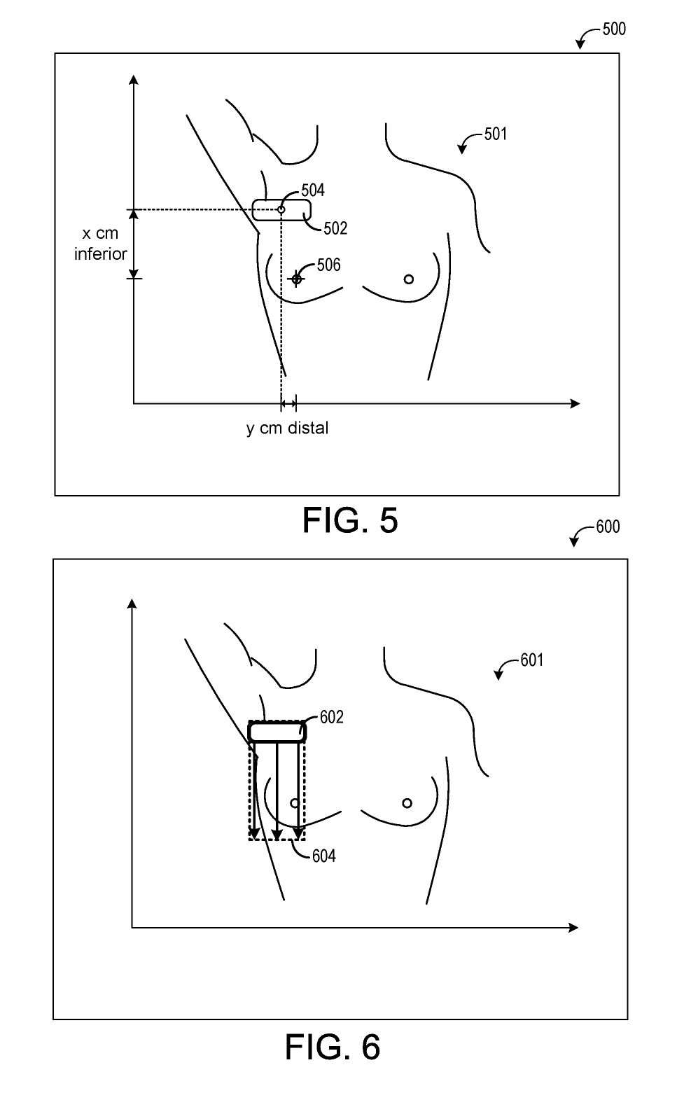

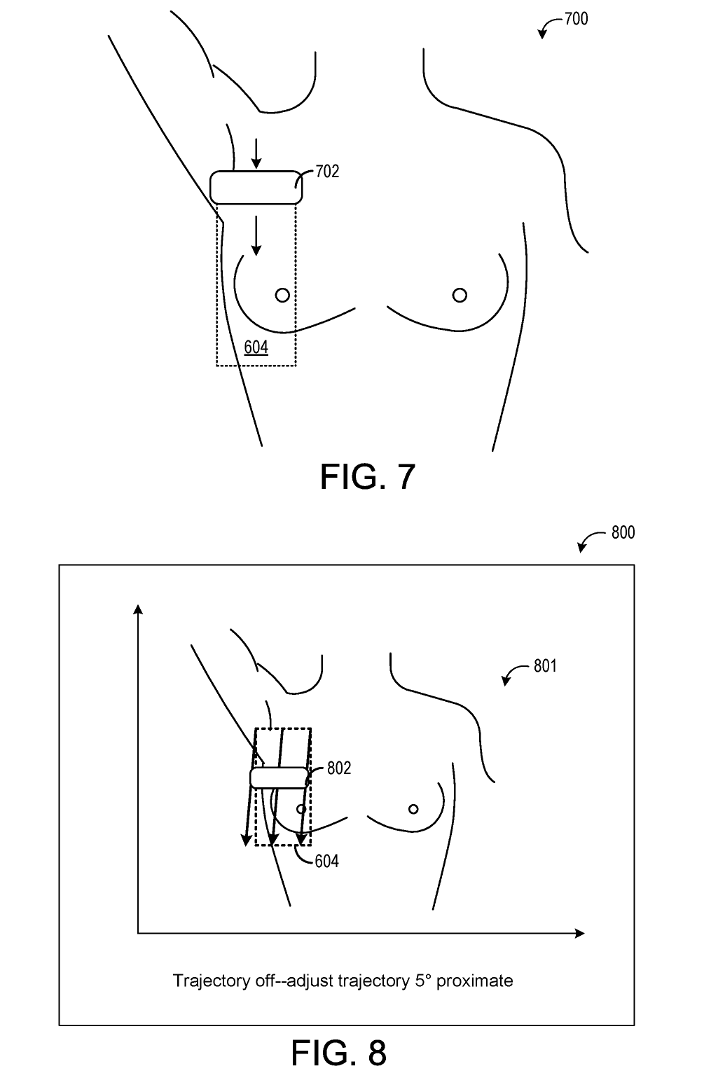

In one example, a hand-held ultrasound ABUS device (HUAD), such as the HUAD depicted in FIGS. 1-2, may include position sensors to track the location of the transducer of the HUAD, such as the sensors depicted in FIG. 3. The HUAD may be used to compress a breast in a generally chestward or head-on direction, or the HUAD may be used on an already compressed, deformed breast, and an operator may move the device to ultrasonically scan the breast. In another example, the HUAD scanning apparatus may compress a breast along planes such as the craniocaudal (CC) plane, the mediolateral oblique (MLO) plane, or the like. The HUAD may include an ultrasound transducer and one or more ultrasound parameter sensors. Information from the ultrasound parameter sensors (which may include position sensors) may be used to provide a semi-automated breast exam. For example, the sensor information may be used to instruct an operator on where to position the HUAD during the exam, how much pressure to apply to the HUAD, and how fast to sweep the HUAD during the exam. The image information acquired during the exam, along with the position sensor information, may be used to construct a three-dimensional volume from which images may be generated. An example method for performing a semi-automated ultrasound exam is illustrated in FIGS. 4A and 4B. Example user interfaces that may be displayed during a semi-automated breast ultrasound exam are illustrated in FIGS. 5, 6, 8, and 9, while example probe sweeps performed by an operator during the semi-automated breast ultrasound exam are illustrated in FIGS. 7 and 10.

Although several examples herein are presented in the particular context of human breast ultrasound, it is to be appreciated that the present teachings are broadly applicable for facilitating ultrasonic scanning of any externally accessible human or animal body part (e.g., abdomen, legs, feet, arms, neck, etc.). Moreover, although several examples herein are presented in the particular context of manual/hand-held scanning (i.e., in which the ultrasound transducer is moved by an operator), it is to be appreciated that one or more aspects of the present teachings can be advantageously applied in a mechanized scanning context (e.g., a robot arm or other automated or semi-automated mechanism).

FIG. 1 illustrates an example hand-held ultrasound device 100 that is configured for performing automated breast ultrasound, referred to herein as a hand-held automated breast ultrasound device (HUAD). HUAD 100 includes an ultrasound probe 101 including housing 102 in which an ultrasound transducer module 104 is positioned. The housing 102 may be shaped to fit in the hand of an operator, and as such may have a length along a horizontal (x) axis (corresponding to a longitudinal axis of the housing) of approximately 10-20 cm, a width along a transverse (z) axis of approximately 5-10 cm, and a height along a vertical (y) axis of approximately 4-8 cm. The housing may have an opening along a bottom surface 106 of the housing, and the transducer module 104 may be positioned across the opening. The bottom surface and transducer array may have a concave curvature (e.g., curving inward toward a top surface of the housing) with a suitable radius of curvature of 0-442 mm.

The probe 101 may comprise an at least partially conformable membrane 108 in a substantially taut state for compressing a breast, the membrane 108 having a bottom surface contacting the breast while the transducer array contacts a top surface thereof to scan the breast. The membrane 108 may be coupled across the opening of the housing. In one example, the membrane is a taut fabric sheet. In other examples, the probe 101 may comprise another suitable acoustic window, such as a plastic window.

The probe 101 may comprise position sensors (not shown in FIG. 1) to allow position and orientation sensing for the transducer. Suitable position sensors (e.g., gyroscopic, magnetic, optical, radio frequency (RF)) may be used. Further, the probe may comprise a display (not shown in FIG. 1) configured to display suitable graphical user interfaces, image data, etc.

A fully functional ultrasound engine for driving an ultrasound transducer and generating volumetric breast ultrasound data from the scans in conjunction with the associated position and orientation information may be coupled to the HUAD, for example the ultrasound engine may be included as part of an ultrasound processor 210 coupled to the probe. The volumetric scan data can be transferred to another computer system for further processing using any of a variety of data transfer methods known in the art. A general purpose computer, which can be implemented on the same computer as the ultrasound engine, is also provided for general user interfacing and system control. The general purpose computer can be a self-contained stand-alone unit, or can be remotely controlled, configured, and/or monitored by a remote station connected across a network.

FIG. 2 is a block diagram 200 schematically illustrating various system components of a HUAD system, including the HUAD probe 101, a display 110, and a scanning processor 210. As illustrated in the embodiment of FIG. 2, the probe 101, display 110, and scanning processor 210 are separate components in communication with each other; however, in some embodiments one or more of the components may be integrated (e.g., the display and scanning processor may be included in a single component).

Referring first to the probe 101, it comprises the transducer module 104 and optionally includes a display 210. Display 210 may be a touch sensitive display configured to receive user input in some examples. In other examples, probe 101 may receive user input via suitable buttons or other user input mechanisms. As explained above with respect to FIG. 1, the transducer module may be positioned within the housing, and the housing and transducer module may be configured to be moved manually by an operator during a sweep of an ultrasound exam.

The transducer module 104 comprises a transducer array 222 of transducer elements, such as piezoelectric elements, that convert electrical energy into ultrasound waves and then detect the reflected ultrasound waves. The transducer module 104 may further include a memory 224. Memory 224 may be a non-transitory memory configured to store various parameters of the transducer module 104, such as transducer usage data (e.g., number of scans performed, total amount of time spent scanning, etc.), as well as specification data of the transducer (e.g., number of transducer array elements, array geometry, etc.) and/or identifying information of the transducer module 104, such as a serial number of the transducer module. Memory 224 may include removable and/or permanent devices, and may include optical memory, semiconductor memory, and/or magnetic memory, among others. Memory 224 may include volatile, nonvolatile, dynamic, static, read/write, read-only, random-access, sequential-access, and/or additional memory. In an example, memory 224 may include RAM. Additionally or alternatively, memory 224 may include EEPROM.

Memory 224 may store non-transitory instructions executable by a controller or processor, such as controller 226, to carry out one or more methods or routines as described herein below. Controller 226 may receive output from various sensors 228 of the transducer module 104 and trigger actuation of one or more actuators and/or communicate with one or more components in response to the sensor output. As will be described in more detail below with reference to FIG. 3, sensors 228 may include one or more position sensors, accelerometers, pressure sensors, strain gauge sensors, and/or temperature sensors. Prior to and/or during scanning, the position of the probe (in six degrees of freedom) may be determined from the output of the position sensor(s) and stored in memory 224 and/or sent to the scanning processor 210. Additionally, in some examples, during scanning, the speed of the probe during scanning may be determined from the output of the accelerometer(s), the pressure across the probe 101 may be measured by the pressure sensors and/or strain gauge sensors, and/or the temperature of the probe 101 may be measured by the temperature sensor(s).

The output from the sensors 228 may be used to provide feedback to an operator of the probe 101 (via user interface 242 of display 110, for example, and/or via a user interface of display 210). For example, the operator may be instructed to reposition the probe prior to initiation of scanning, if the probe is not located at a predetermined position. In another example, the operator may be instructed to adjust an angle, speed, and/or location of probe during scanning. In a still further example, if the pressure distribution across the transducer module is not equal, a user may be notified to reposition the probe 101, increase or decrease compression of the probe, etc.

Probe 101 may be in communication with scanning processor 210, to send raw scanning data to an image processor, for example. Additionally, data stored in memory 224 and/or output from sensors 228 may be sent to scanning processor 210 in some examples. Further, various actions of the probe 101 (e.g., activation of the transducer elements) may be initiated in response to signals from the scanning processor 210. Probe 101 may optionally communicate with display 110 and/or display 210, in order to notify a user to reposition the probe, as explained above, or to receive information from a user (via user input 244), for example.

Turning now to scanning processor 210, it includes an image processor 212, storage 214, display output 216, and ultrasound engine 218. Ultrasound engine 218 may drive activation of the transducer elements of the transducer array 222 of transducer module 104. Further, ultrasound engine 218 may receive raw image data (e.g., ultrasound echoes) from the probe 101. The raw image data may be sent to image processor 212 and/or to a remote processor (via a network, for example) and processed to form a displayable image of the tissue sample. It is to be understood that the image processor 212 may be included with the ultrasound engine 218 in some embodiments.

Information may be communicated from the ultrasound engine 218 and/or image processor 212 to a user of the HUAD system via the display output 216 of the scanning processor 210. In one example, the user of the HUAD system may include an ultrasound technician, nurse, or physician such as a radiologist. For example, processed images of the scanned tissue may be sent to the display 110 via the display output 216. In another example, information relating to parameters of the scan, such as the progress of the scan, may be sent to the display 110 via the display output 216. The display 110 may include a user interface 242 configured to display images or other information to a user. Further, user interface 242 may be configured to receive input from a user (such as through user input 244) and send the input to the scanning processor 210. User input 244 may be a touch screen of the display 110 in one example. However, other types of user input mechanisms are possible, such as a mouse, keyboard, etc.

Scanning processor 210 may further include storage 214. Similar to memory 224, storage 214 may include removable and/or permanent devices, and may include optical memory, semiconductor memory, and/or magnetic memory, among others. Storage 214 may include volatile, nonvolatile, dynamic, static, read/write, read-only, random-access, sequential-access, and/or additional memory. Storage 214 may store non-transitory instructions executable by a controller or processor, such as ultrasound engine 218 or image processor 212, to carry out one or more methods or routines as described herein below. Storage 214 may store raw image data received from the ultrasound probe, processed image data received from image processor 212 or a remote processor, and/or additional information.

FIG. 3 shows a cross-section of the transducer module 104. Specifically, FIG. 3 shows a schematic 300 of a front cross-section of the transducer module 104 in a plane defined by the vertical axis and the horizontal axis. The transducer module includes a bottom surface 106 configured to contact a patient tissue during scanning (via a membrane in some examples). Positioned in the transducer module 104, near the bottom surface, are a plurality of transducer elements 302 forming the transducer array 222. As illustrated, the transducer elements 302 are arranged in groups that are equally spaced apart from each other across the entire length of the contact end. However, other configurations for the transducer elements 302 are possible. For example, the transducer elements may be arranged individually. While a single row of transducer elements 302 are illustrated in FIG. 3, it is to be understood that at least in some embodiments, additional transducer elements may extend across a width of the transducer module 104 in order to form an array of transducer elements.

The transducer elements 302 may be positioned a distance from the surface (e.g., contact surface) of the bottom surface 106 of the transducer module 104. This distance may be the same for all transducer elements, such that if the surface of the transducer module is curved, the array of transducer elements 302 is also curved. However, in other embodiments, this distance may differ for transducer elements positioned in different regions of the transducer module 104. For example, the transducer elements 302 may be arranged in a straight row without curvature that extends across a length of the transducer module 104. If the bottom surface 106 is curved, the transducer elements 302 located along each side of the transducer module 104 may be spaced a farther distance from the surface than the transducer elements located in the center of the transducer module 104. Additionally, the array may include one or more mechanical focusing elements, such as acoustic lenses, along the length of the transducer module 104 and positioned between the transducer elements 302 and the bottom surface 106.

Further, the transducer elements 302 may be positioned across the entire length and width of the transducer module 104, or the transducer elements 302 may be positioned across only a portion of the length and/or width of the transducer module 104. For example, the transducer elements 302 may extend only across a central area of the transducer module.

Each transducer element is configured to transmit and receive ultrasound waves to acquire image data of the tissue being scanned. In order to send the image data to a processor for image processing, each transducer element may be connected to a cable or other connection. In this way, the raw image data collected by the transducer module may be sent to an image processor via the connection with the module receiver.

Further, the plurality of sensors 228, including sensor 304, may be distributed across the transducer module 104. The sensors may include one or more position sensors, accelerometers, pressure sensors, strain gauge sensors, and/or one or more temperature sensors. The position sensors may be configured to measure position of the probe in six degrees of freedom (e.g., pitch, roll, and yah), and may include gyroscopes, optical position sensors, electromagnetic position sensors, or other suitable sensor configuration. Additionally or alternatively, the probe may include one or more inertial measurement units (IMUs) that include accelerometer(s) and gyroscope(s). The sensors may be distributed evenly across the transducer module 104, as shown, or other suitable arrangement. In one example, the sensors are positioned proximate to the bottom surface 106 of the transducer module 104. The output from the sensors may be stored in the memory 224 of the transducer module 104.

In one example, the transducer module 104 is a linear array transducer comprising 768 piezoelectric elements. In alternate embodiments, the transducer module 104 may include more or less than 768 transducer elements. In one example, an operating frequency of the transducer array is in a range from 2 MHz to 15 MHz. In another example, the operating frequency range may be from 6 MHz to 10 MHz. In yet another example, the operating frequency may be 7.5 MHz. The bottom surface 106 of the transducer module 104 may also include mechanical focusing elements, such as acoustic lenses, for focusing the ultrasound waves. The transducer elements of the transducer array may be spaced along a length of the transducer module 104.

The length of the transducer module 104 is in a range from approximately 10 cm to 20 cm. In one example, the length of the transducer module 104 is 15 cm. In another example, the length of the transducer module is 18 cm. Different transducer modules 104 may have different lengths for differently sized patients and based on a size of the target tissue area for scanning. For example, the length may be sized in order to allow imaging of a breast in two or three horizontal sweeps.

As shown in FIG. 3, the bottom surface of the transducer module 104 is curved. As a result, the transducer array may also be curved. However, in other examples the transducer array may not be curved and mechanical focusing elements may be used to focus the sound waves. The bottom surface has a curvature radius. In some embodiments, a transducer module may have a curvature radius of substantially zero such that the bottom surface is substantially flat. In other examples, the curvature radius may be based on a patient's anatomy or tissue contour (e.g., convexity).

The HUAD may be configured (e.g., shaped) to fit comfortably in the operator's hand and may include ergonomic concessions to provide the comfortable fitting. The HUAD may be wider than it is tall to minimize the degrees of transducer module roll as the operator translates the HUAD over the breast or body. One degree of transducer module roll at the skin surface may be compounded as the ultrasound penetrates the tissue. Wireless position sensors, accelerometers, and other electronic clusters such as strain gauges, are embedded inside the HUAD to provide position information (with six degrees of freedom), speed information, direction of movement information, and compression amount information.

Turning now to FIGS. 4A-4B, a method 400 for generating images based on data acquired by a HUAD is illustrated. Method 400 may be executed by a computing device, such as scanning processor 210, according to instructions stored in memory thereon, in conjunction with a hand-held ultrasound probe, such as probe 101. At 402, method 400 optionally includes instructing a user to mark one or more fiducial markers on a subject to be imaged. The operator may be instructed to mark the fiducial(s) via instructions displayed via a user interface displayed on a display device associated with the computing device, for example. The fiducial(s) may include the location(s) of relevant anatomy that may be used to orient and/or stitch together volume data acquired by the ultrasound probe. For example, during automated breast ultrasound, the nipple, sternum, and/or other relevant anatomy may be defined as fiducial markers.

At 404, method 400 includes determining the location of the fiducial marker(s) based on output from one or more position sensors of the ultrasound probe. For example, an operator may position the ultrasound probe over the nipple and enter an input (e.g., enter a user input to the ultrasound probe via a button or touch screen, apply extra pressure to the ultrasound probe, or temporarily lift the ultrasound probe off the subject) indicating the probe is positioned over the nipple. The computing device may store the position sensor output when the location of the nipple is indicated. The location of the nipple may be an absolute position (e.g., relative to a coordinate system) or the location of the nipple may be a relative position (e.g., the position data may be set to zero at the nipple, thus allowing any other collected position data to be relative to the location of the nipple). The location of other fiducial markers (e.g., the sternum) may be determined in a similar fashion.

At 406, method 400 optionally includes instructing the operator to position the ultrasound probe at a first location relative to the fiducial. For example, a user interface may display instructions guiding the operator to position the ultrasound probe at the first location. The first location may be a suitable location, such as a predetermined distance directly inferior the nipple, a predetermined distance at a given angle (or clock position) relative to the nipple, or other location. The computing device may receive output from the position sensor(s) while the operator is positioning the ultrasound probe, and the computing device may instruct the operator to position the ultrasound probe based on the output from the position sensor(s). For example, the computing device may determine that the probe is positioned two cm to the right of the first location and then output instructions to the user to move the probe two cm to the right.

FIG. 5 shows an example user interface 500 that may be output (e.g., for display via a display device, such as display 110 or display 210) during a semi-automated breast ultrasound exam. User interface 500 may include a set of graphics 501 that visually indicates the probe 502, a location marker 504 of the probe (which may be a location of a position sensor in one example), and a fiducial marker 506. The set of graphics 501 further includes a coordinate system. As shown, the graphics also include a depiction (e.g., image) of the patient/tissue to be imaged. Such a depiction may be a real-time image, a stock image, or the depiction may be dispensed with.

As shown by user interface 500, the operator is being instructed to position the probe such that the location marker 504 is at a predetermined first position relative to the fiducial marker. Herein, the first position includes the location marker being positioned a distance (e.g., x cm) inferior the fiducial marker and a distance (e.g., y cm) distal the fiducial marker. However, other positions relative the fiducial marker are possible, such as a distance and a clock position (e.g., x cm and 11 o'clock). In some examples, the depicted location of the probe may reflect the actual position of the probe. In other examples, the depicted location of the probe may be fixed at the first position and may not reflect the actual position of the probe.

The user interface 500 may include instructions that are updated as the operator moves the probe position. For example, as the probe is moved by the operator, the depicted location of probe may change to reflect the updated location of the probe. Additional or alternative instructions may be displayed, such as text that guides the operator to the first position, e.g., “move the probe 1 cm distal.” Additionally, once the probe is positioned at the first position, a notification may be output to the operator. For example, FIG. 6 shows another example user interface 600 that may be displayed once the probe has been positioned at the first position. User interface 600 includes a set of graphics 601 that includes the depiction (e.g., image) of the patient/tissue to be imaged and a depiction of the probe 602. The probe 602 includes a visual marker (e.g., highlighting) to indicate to the operator that the actual probe is positioned at the first position. Additionally, a first region 604 to be imaged is depicted along with a target trajectory (indicated by the solid arrows). In this way, the operator may be instructed to commence the first sweep along the target trajectory. Other information may also be displayed, such as target sweep speed.

Returning to FIG. 4A, at 408, method 400 includes receiving first ultrasound image data during a first sweep of the ultrasound probe. The received image data may include ultrasound echoes of ultrasound waves transmitted by the transducer elements of the transducer array of the probe. The ultrasound echoes may be sent to an image processor to be processed into an image of the tissue. In some examples, the image data may include volumetric ultrasound data. At 410, method 400 includes receiving first position and/or speed data of the ultrasound probe during the first sweep. The position sensor(s) and accelerometer(s) may be periodically sampled over the course of the first sweep, and the sampled output may be sent to the computing device. The first position and/or speed data may be stored in memory of the computing device. In one example, each frame of image data received by the computing device may have position and/or speed data associated with that frame. Further, in some examples, instructions may be output to the operator of the ultrasound probe based on the output from the position sensors, strain gauge sensors, and/or accelerometers. For example, the speed of the probe during the first sweep may be determined from the accelerator data, and if the speed is greater than a threshold, the operator may be instructed to slow the speed of the sweep. Likewise, the angle and/or position of the probe may be determined over the course of the first sweep, and the operator may be instructed to adjust the position of the probe if the probe angle differs from a desired angle, if the trajectory of the probe diverges from a target trajectory during the first sweep, etc. Further, the output from the strain gauge sensor(s) may be used to provide instructions to the operator in order to maintain a desired and/or consistent compression of the tissue.

FIG. 7 shows an example first sweep 700 of an ultrasound probe 702 (e.g., probe 101) during a semi-automated breast ultrasound exam. As shown, the probe 702 is being swept by an operator (not shown in FIG. 7) in a direction indicated by the arrows, in order to collect image data of the first region 604 of a patient (in one example, the image data collected during the first sweep may be referred to as a first acquisition data set). As explained above, the sweep may commence once the operator determines the probe is in the first position. Further, the operator may adjust sweep speed, compression, trajectory, and/or other parameters during the sweep based on feedback from the probe sensors.

At 412, method 400 determines if one or more sweep quality parameters have been met. The sweep quality parameters may include a speed of the probe during the first sweep not exceeding a predetermined speed, a trajectory of the probe during the first sweep tracking a desired trajectory, sufficient quality image, speed, and/or position data having been acquired during the first sweep, or other suitable quality parameters. If the sweep quality parameters have not been met, method 400 proceeds to 414 to optionally instruct the operator to change one or more sweep parameters, such as the sweep trajectory, initial or final position of the probe during the sweep, sweep speed, etc. Method 400 then returns to 406 or 408, so that another first sweep may be performed.

As explained above, the operator may be instructed to adjust sweep speed, trajectory, compression, and/or other sweep parameters during the sweep. By providing real-time feedback to the operator, high quality sweeps (e.g., meeting all the sweep quality parameters) may be obtained without multiple sweeps of the same tissue region. FIG. 8 shows an example user interface 800 that may be displayed during the first sweep. User interface 800 includes a set of graphics 801 that includes the depiction of the subject being imaged, a depiction of the probe 802, the first region 604 to be imaged, and a calculated trajectory (shown by the solid arrows). As appreciated by FIG. 8, the projected trajectory has deviated from the target trajectory. If the sweep were allowed to progress along the projected trajectory, some tissue within the first region 604 may not be imaged, resulting in a low-quality volume, repeated sweeps, or other adjustments that may prolong imaging or reduce image quality. Thus, when a deviation from the target trajectory is detected, the operator may be notified via the user interface. As shown, text instructions guiding the operator to adjust the trajectory are displayed within user interface 800. Instructions to make other adjustments may additionally or alternatively be displayed, such as compression amount, sweep speed, etc.

Returning to FIG. 4A, if the sweep quality parameters have been met, method 400 proceeds to 416 to optionally instruct the operator to position the ultrasound probe at a second location. The second location may be relative to the fiducial marker, or the second location may be relative to the first location. For example, the second location may be a predetermined distance to the right or to the left of the first position. In some examples, the second position may be selected such that the probe partially overlaps with the position of the probe while in the first position, such that a portion of the anatomy imaged during the first sweep is also imaged during the second sweep.

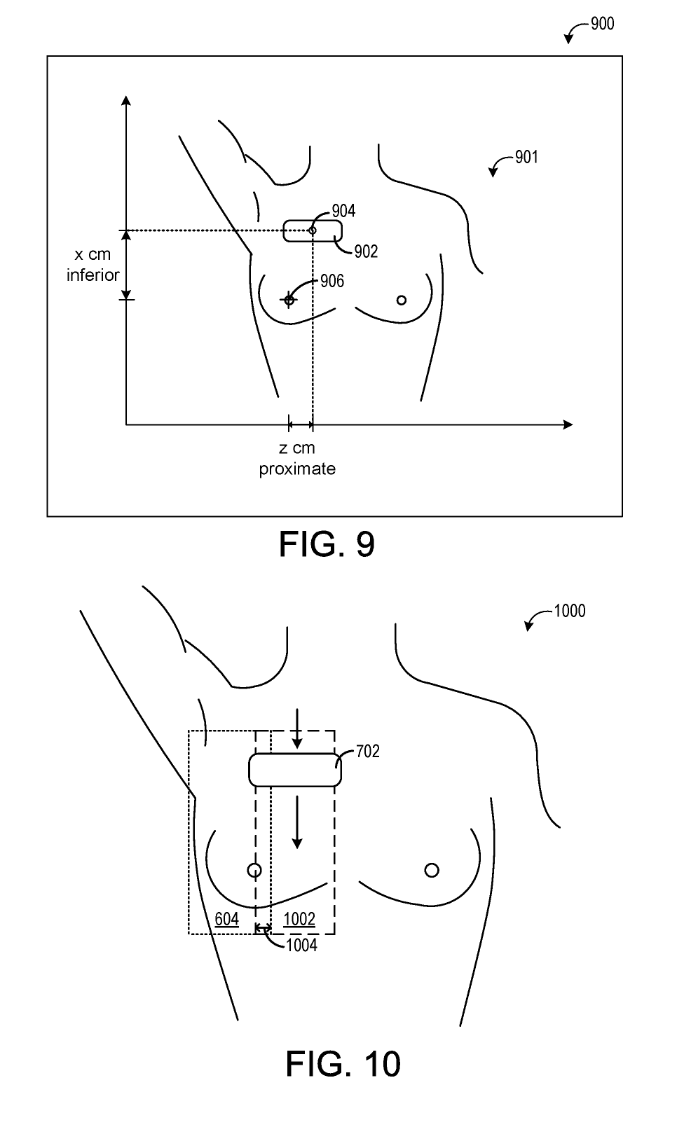

FIG. 9 shows another example user interface 900 that may be output (e.g., for display via a display device, such as display 110 or display 210) during a semi-automated breast ultrasound exam. User interface 900 may include a set of graphics 901 that visually indicates the probe 902, a location marker 904 of the probe (which may be a location of a position sensor in one example), and a fiducial marker 906. The set of graphics 901 further includes a coordinate system. As shown, the graphics also include a depiction (e.g., image) of the patient/tissue to be imaged. Such a depiction may be a real-time image, a stock image, or the depiction may be dispensed with.

As shown by user interface 900, the operator is being instructed to position the probe such that the location marker 904 is at a predetermined second position relative to the fiducial marker. Herein, the first position includes the location marker being positioned a distance (e.g., x cm) inferior the fiducial marker and a distance (e.g., z cm) proximate the fiducial marker. However, other positions relative the fiducial marker are possible, such as a distance and a clock position (e.g., x cm and 1 o'clock). In some examples, the depicted location of the probe may reflect the actual position of the probe. In other examples, the depicted location of the probe may be fixed at the first position and may not reflect the actual position of the probe.

The user interface 900 may include instructions that are updated as the operator moves the probe position. For example, as the probe is moved by the operator, the depicted location of probe may change to reflect the updated location of the probe. Additional or alternative instructions may be displayed, such as text that guides the operator to the first position, e.g., “move the probe 1 cm proximate.” Additionally, once the probe is positioned at the second position, a notification may be output to the operator.

At 418, method 400 includes receiving second ultrasound image data during a second sweep of the ultrasound probe, and at 420, includes receiving second position and/or speed data of the ultrasound probe during the second sweep. FIG. 10 shows an example second sweep 1000 of the ultrasound probe 702 during a semi-automated breast ultrasound exam. As shown, the probe 702 is being swept by the operator (not shown in FIG. 10) in a direction indicated by the arrows, in order to collect image data of a second region 1002 of the patient (in one example, the image data collected during the second sweep may be referred to as a second acquisition data set). As explained above, the sweep may commence once the operator determines the probe is in the second position. Further, the operator may adjust sweep speed, compression, trajectory, and/or other parameters during the sweep based on feedback from the probe sensors. Additionally, as shown in FIG. 10, an overlap region 1004 may include tissue imaged during both the first sweep and the second sweep.

At 422, method 400 determines if sweep quality parameters have been met for the second sweep. If not, method 400 proceeds to 424 to instruct the operator to change one or more sweep parameters, and then method 400 loops back to 416 or 418 to perform another second sweep. If the sweep quality parameters are met, method 400 proceeds to 426 to determine if the second sweep was the final sweep indicated for the exam, of if additional sweeps are indicated. If additional sweeps are indicated, method 400 proceeds to 428 repeat the positioning instructions of the ultrasound probe and data acquisition (e.g., image, position, and/or speed data), and then loops back to 426.

If no additional sweeps are indicated, method 400 proceeds to 430 (illustrated in FIG. 4B) to project the first ultrasound image data onto a volume using the first position sensor output. The volume may include a three-dimensional array of voxels. The first ultrasound image data may include intensity (and other parameters, such as opacity) for each voxel of a subset of the voxels corresponding to the location of the first sweep. Appropriate ultrasound image data may be projected onto a given voxel based on the position of the probe when that ultrasound image data was acquired. At 432, method 400 includes projecting the second ultrasound image data onto the volume using the second position sensor output, and at 434, method 400 includes projecting additional ultrasound image data onto the volume using the additional position sensor output.

For example, a computing device (e.g., scanning processor 210) may analyze the precise location of, and all anatomical structural details within, every pixel of the acquired image data. In addition, the computing device may calculate the HUAD speed and movement direction using the embedded sensors. The image data is consolidated along an elevation plane of the transducer module as the operator moves the HUAD over the tissue, using a suitable volume generation mechanism, such as LOGIQ View. The consolidated images from one linear sweep are referred to as an acquisition data set. The computing device compares the acquired image data, pixel by pixel, from the nearest adjacent acquisition data set and stiches the acquired image data from different sweeps together into one consolidated image volume. In one example, the computing device may detect one or more anatomical features of the subject in each acquisition data set and, and mark each detected anatomical feature as a respective fiducial marker. Example anatomical features that may be detected and/or used as fiducial markers include the nipple, the chest wall, speckle characteristics, hyperechoic architectures, and hypoechoic regions. In some examples, the use of convoluted neural networks may aid and/or improve feature detection and classification, as permitted by algorithm performance and system features. Non-rigid image registration may then be performed to stitch the acquisition data sets together into the consolidated image volume. The non-rigid image registration may register the acquisition data sets using the detected fiducial markers. For example, a first acquisition data set may be used as the reference image or data set. The nipple may be detected in the first acquisition data set. The nipple may also be detected in a second acquisition data set. The second acquisition data set may be registered with the first acquisition data set by aligning the nipple in the two acquisition data sets. The position sensor information may be used to aid or enhance this registration, for example by aligning acquisition data sets that do not include fiducial markers, by resolving conflicts or uncertainties between acquisition data sets, by defining a region of interest where the anatomical feature is likely to be located (in order to expedite the detection of the anatomical feature), etc. In one example, the position sensor information may be used to identify a region of a first acquisition data set that overlaps a region of the second acquisition data set (e.g., an overlap region) and stitch together the two acquisition data sets by align the two acquisition data sets along the overlap region.

At 436, method 400 determines if the volume registration is accurate. As described above, image data acquired from multiple sweeps of the probe is projected onto a single volume. Due to overlapping sweeps, some voxels of the volume will be populated with image data from more than one sweep (for example, the second sweep illustrated in FIG. 10 includes some image data of tissue that was also imaged during the first sweep, as shown by the overlap region). If positional inaccuracies, differences in image data quality, or other irregularities occur among sweeps, smooth registration of the image data from the different sweeps on the same volume may not occur. Inaccurate registration may lead to poor quality images reconstructed from the data volume, false negative or false positive lesion detection, or other issues.

Thus, at least in one example, inaccurate registration may be determined by comparing the ultrasound data intensity values for overlapping voxels that are populated with image data from both the first sweep and second sweep. For example, during the projection of the first image data onto the volume, a first voxel may be populated with intensity information from the first sweep. Then, during the projection of the second image data onto the volume, that same first voxel may also be populated with intensity information from the second sweep. If the intensity information form the first sweep is different than the intensity information form the second sweep, it may be determined that inaccurate registration at that voxel location has occurred. If a threshold number of overlapping voxels receive different intensity information from different sweeps, inaccurate registration of the entire volume may be indicated.

If it is determined that the volume registration is accurate, method 400 proceeds to 438 to generate one or more images from the full data volume. The images may be generated according to a suitable mechanism, such as ray-casting, intensity projection, etc. At 440, method 400 includes displaying and/or saving the generated images. In particular, at least in some examples, the images may be saved with identifying positional information that indicates the plane of the image and distance/position relative to the fiducial marker. By doing so, future ultrasound exams may be conducted and images of the same location taken over each exam may be compared, to facilitate accurate compare-to-prior workflow exams. Method 400 then returns.

If at 436 it is determined that the volume registration is not accurate, method 400 proceeds to 442 to project each of the first and second image data, and each additional image data, on separate volumes. At 444, method 400 displays a representation of each separate volume on a display device, and 446, method 400 generates one or more images from selected data volume(s). The generated images are then displayed and/or saved, along with associated position information, at 448, similar to the displaying and/or saving at 440. In this way, images may be generated from separate volumes, according to user selection, rather than generating images from a single, common volume.

Thus, the methods and systems described herein provide for a hand-held ultrasound probe that includes position and speed sensors to allow for intelligent guidance of the ultrasound probe. By doing so, precise, repeatable sweeps of the probe may be performed by an operator, and the image data acquired during each sweep of the probe may be reconstructed into images from a three-dimensional volume that is generated from the image data using the position data. Further, due to the inclusion of the position sensor information along with the image data, images from a desired plane may be generated, aligned, and/or otherwise manipulated without requiring a predetermined region of interest (e.g., a nipple) be located in the images.

The technical effect of performing an automated ultrasound exam using a hand-held ultrasound probe is the generation of images in multiple planes from a three-dimensional volume while reducing the cost and complexity of the ultrasound probe.

An example relates to a system for ultrasonically scanning a tissue sample. The system includes a hand-held ultrasound probe including a housing and a transducer module comprising a transducer array of transducer elements; one or more position sensors coupled within the housing; and a controller configured to generate one or more images based on ultrasound data acquired by the transducer module and further based on position sensor data collected by the one or more position sensors.

The housing may define an opening, and the system may further include a membranous sheet disposed across the opening, the transducer module positioned to contact the membranous sheet.

In an example, to generate the one or more images based on the ultrasound data, the controller is configured to associate each frame of the ultrasound data with position sensor data indicating a position of the ultrasound probe when that frame of ultrasound data was acquired; generate a three-dimensional volume from each frame and associated position sensor data; and generate the one or more images from the three-dimensional volume.

In an example, to generate the three-dimensional volume, the controller is configured to consolidate ultrasound data from each frame of a first linear sweep of the ultrasound probe along an elevation plane of the transducer array as the ultrasound probe is moved over a subject being imaged in order to generate a first acquisition data set; consolidate ultrasound data from each frame of a second linear sweep of the ultrasound probe along the elevation plane of the transducer array as the ultrasound probe is moved over the subject in order to generate a second acquisition data set; and stitch together first acquisition data set and second acquisition data set to form the three-dimensional volume. In an example, to stitch together the first acquisition data set and the second acquisition data set, the controller is configured to detect one or more anatomical features of the subject in the first acquisition data set and second acquisition data set, and mark each detected anatomical feature as a respective fiducial marker; and stitch together the first acquisition data set and second acquisition data set via a non-rigid image registration protocol using the respective fiducial markers.

In an example, the system further includes one or more accelerometers coupled within the housing. The controller may be configured to output instructions guiding an operator of the ultrasound probe to adjust one or more of a speed and position of the ultrasound probe based on output from one or more of the one or more accelerometers and one or more position sensors.

In an example, the system further includes one or more strain gauge sensors coupled within the housing. The controller may be further configured to output instructions guiding an operator of the ultrasound probe to adjust compression of the ultrasound probe based on output from the one or more strain gauge sensors.

In an example, the system further includes a display device coupled to the housing.

An example relates to method for an ultrasound imaging device including a hand-held ultrasound probe. The method includes receiving first image data from the ultrasound probe during a first sweep of a subject with the ultrasound probe, the first sweep initiated from a first predetermined location; receiving first position data from one or more position sensors of the ultrasound probe during the first sweep; receiving second image data from the ultrasound probe during a second sweep of the subject with the ultrasound probe, the second sweep initiated from a second predetermined location; receiving second position data from the one or more position sensors during the second sweep; and generating an image of the subject with the first image data and second image data and further based on the first position information and the second position information.

In an example, generating the image of the subject with the first image data and the second image data and further based on the first position information and the second position information includes associating each frame of the first image data with corresponding first position information indicating a position of the ultrasound probe when that frame of image data was acquired; associating each frame of the second image data with corresponding second position information indicating a position of the ultrasound probe when that frame of image data was acquired; projecting each frame of the first image data and each frame of the second image data onto a common three-dimensional volume based on the corresponding first or second position information; and generating the image from the three-dimensional volume.

In an example, the method includes saving the image in memory along with associated ultrasound probe position information.

In an example, generating the image of the subject with the first image data and second image data and further based on the first position information and the second position information includes associating each frame of the first image data with corresponding first position information indicating a position of the ultrasound probe when that frame of image data was acquired; associating each frame of the second image data with corresponding second position information indicating a position of the ultrasound probe when that frame of image data was acquired; projecting each frame of the first image data onto a first three-dimensional volume based on the corresponding second position information and projecting each frame of the second image data onto a second three-dimensional volume based on the corresponding second position information; and generating the image from the first three-dimensional volume or the second three-dimensional model.

In an example, the method further includes receiving a user input indicative of a fiducial marker and determining a location of the fiducial marker based on output from the one or more position sensors. The method may further include outputting instructions to guide an operator to position the ultrasound probe at the first location, and wherein the first location is relative to the fiducial marker.

An example relates to method for an ultrasound imaging device including a hand-held ultrasound probe. The method includes receiving an indication of a location of a region of interest of a subject to be imaged, the indication based at least in part on output from one or more position sensors positioned on the ultrasound probe; providing first feedback to an operator of the ultrasound imaging device to position the ultrasound probe at a first predetermined location relative to the location of the region of interest; receiving first image data from the ultrasound probe during a first sweep of the subject with the ultrasound probe; receiving first position data from the one or more position sensors during the first sweep; providing second feedback to the operator to position the ultrasound probe at a second predetermined location relative to the location of the region of interest; receiving second image data from the ultrasound probe during a second sweep of the subject with the ultrasound probe; receiving second position data from the one or more position sensors during the second sweep; and generating an image of the subject with the first image data and second image data and further based on the first position information and the second position information.

In an example, the first sweep partially overlaps the second sweep in an overlap region such that the first image data and second image data each include overlap image data corresponding to the overlap region, and the method further includes determining a registration accuracy of the first image data relative to the second image data by comparing the overlap image data of the first image data to the overlap image data of the second image data.

In an example, when the registration accuracy is greater than a threshold, the method further includes associating each frame of the first image data with corresponding first position information indicating a position of the ultrasound probe when that frame of image data was acquired; associating each frame of the second image data with corresponding second position information indicating a position of the ultrasound probe when that frame of image data was acquired; projecting each frame of the first image data and each frame of the second image data onto a common three-dimensional volume based on the corresponding first or second position information; and generating the image from the three-dimensional volume.

In an example, when the registration accuracy is not greater than a threshold, the method further includes associating each frame of the first image data with corresponding first position information indicating a position of the ultrasound probe when that frame of image data was acquired; associating each frame of the second image data with corresponding second position information indicating a position of the ultrasound probe when that frame of image data was acquired; projecting each frame of the first image data onto a first three-dimensional volume based on the corresponding second position information and projecting each frame of the second image data onto a second three-dimensional volume based on the corresponding second position information; and generating the image from the first three-dimensional volume or the second three-dimensional model.

In an example, the method further includes displaying the image of the subject on a display device.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A system for ultrasonically scanning a tissue sample, comprising:
a hand-held ultrasound probe including a housing and a transducer module comprising a transducer array of transducer elements;
one or more position sensors coupled within the housing; and
a controller configured to generate one or more images based on ultrasound data acquired by the transducer module and further based on position sensor data collected by the one or more position sensors.

2. The system of claim 1, wherein the housing defines an opening and further comprising a membranous sheet disposed across the opening, the transducer module positioned to contact the membranous sheet.

3. The system of claim 1, wherein to generate the one or more images based on the ultrasound data, the controller is configured to:
associate each frame of the ultrasound data with position sensor data indicating a position of the ultrasound probe when that frame of ultrasound data was acquired;
generate a three-dimensional volume from each frame and associated position sensor data; and
generate the one or more images from the three-dimensional volume.

4. The system of claim 3, wherein to generate the three-dimensional volume, the controller is configured to:
consolidate ultrasound data from each frame of a first linear sweep of the ultrasound probe along an elevation plane of the transducer array as the ultrasound probe is moved over a subject being imaged in order to generate a first acquisition data set;
consolidate ultrasound data from each frame of a second linear sweep of the ultrasound probe along the elevation plane of the transducer array as the ultrasound probe is moved over the subject in order to generate a second acquisition data set; and
stitch together the first acquisition data set and the second acquisition data set to form the three-dimensional volume.

5. The system of claim 4, wherein to stitch together the first acquisition data set and the second acquisition data set, the controller is configured to:
detect one or more anatomical features of the subject in the first acquisition data set and second acquisition data set, and mark each detected anatomical feature as a respective fiducial marker; and
stitch together the first acquisition data set and second acquisition data set via a non-rigid image registration protocol using the respective fiducial markers.

6. The system of claim 1, further comprising one or more accelerometers coupled within the housing.

7. The system of claim 6, wherein the controller is further configured to output instructions guiding an operator of the ultrasound probe to adjust one or more of a speed and position of the ultrasound probe based on output from one or more of the one or more accelerometers and one or more position sensors.

8. The system of claim 1, further comprising one or more strain gauge sensors coupled within the housing.

9. The system of claim 8, wherein the controller is further configured to output instructions guiding an operator of the ultrasound probe to adjust compression of the ultrasound probe based on output from the one or more strain gauge sensors.

10. A method for an ultrasound imaging device including a hand-held ultrasound probe, comprising:
receiving first image data from the ultrasound probe during a first sweep of a subject with the ultrasound probe, the first sweep initiated from a first predetermined location;
receiving first position data from one or more position sensors of the ultrasound probe during the first sweep;
receiving second image data from the ultrasound probe during a second sweep of the subject with the ultrasound probe, the second sweep initiated from a second predetermined location;
receiving second position data from the one or more position sensors during the second sweep; and
generating an image of the subject with the first image data and second image data and further based on the first position information and the second position information.

11. The method of claim 10, wherein generating the image of the subject with the first image data and the second image data and further based on the first position information and the second position information comprises:
associating each frame of the first image data with corresponding first position information indicating a position of the ultrasound probe when that frame of image data was acquired;
associating each frame of the second image data with corresponding second position information indicating a position of the ultrasound probe when that frame of image data was acquired;
projecting each frame of the first image data and each frame of the second image data onto a common three-dimensional volume based on the corresponding first or second position information; and
generating the image from the three-dimensional volume.

12. The method of claim 11, further comprising saving the image in memory along with associated ultrasound probe position information.

13. The method of claim 10, wherein generating the image of the subject with the first image data and second image data and further based on the first position information and the second position information comprises:
associating each frame of the first image data with corresponding first position information indicating a position of the ultrasound probe when that frame of image data was acquired;
associating each frame of the second image data with corresponding second position information indicating a position of the ultrasound probe when that frame of image data was acquired;
projecting each frame of the first image data onto a first three-dimensional volume based on the corresponding second position information and projecting each frame of the second image data onto a second three-dimensional volume based on the corresponding second position information; and
generating the image from the first three-dimensional volume or the second three-dimensional model.

14. The method of claim 10, further comprising receiving a user input indicative of a fiducial marker and determining a location of the fiducial marker based on output from the one or more position sensors.

15. The method of claim 14, further comprising outputting instructions to guide an operator to position the ultrasound probe at the first location, and wherein the first location is relative to the fiducial marker.

16. A method for an ultrasound imaging device including a hand-held ultrasound probe, comprising:
receiving an indication of a location of a region of interest of a subject to be imaged, the indication based at least in part on output from one or more position sensors positioned on the ultrasound probe;
providing first feedback to an operator of the ultrasound imaging device to position the ultrasound probe at a first predetermined location relative to the location of the region of interest;
receiving first image data from the ultrasound probe during a first sweep of the subject with the ultrasound probe;
receiving first position data from the one or more position sensors during the first sweep;
providing second feedback to the operator to position the ultrasound probe at a second predetermined location relative to the location of the region of interest;
receiving second image data from the ultrasound probe during a second sweep of the subject with the ultrasound probe;
receiving second position data from the one or more position sensors during the second sweep; and
generating an image of the subject with the first image data and second image data and further based on the first position information and the second position information.

17. The method of claim 16, wherein the first sweep partially overlaps the second sweep in an overlap region such that the first image data and second image data each include overlap image data corresponding to the overlap region, and further comprising determining a registration accuracy of the first image data relative to the second image data by comparing the overlap image data of the first image data to the overlap image data of the second image data.

18. The method of claim 17, wherein when the registration accuracy is greater than a threshold, the method further comprises:
associating each frame of the first image data with corresponding first position information indicating a position of the ultrasound probe when that frame of image data was acquired;
associating each frame of the second image data with corresponding second position information indicating a position of the ultrasound probe when that frame of image data was acquired;
projecting each frame of the first image data and each frame of the second image data onto a common three-dimensional volume based on the corresponding first or second position information; and
generating the image from the three-dimensional volume.

19. The method of claim 17, wherein when the registration accuracy is not greater than a threshold, the method further comprises:
associating each frame of the first image data with corresponding first position information indicating a position of the ultrasound probe when that frame of image data was acquired;
associating each frame of the second image data with corresponding second position information indicating a position of the ultrasound probe when that frame of image data was acquired;
projecting each frame of the first image data onto a first three-dimensional volume based on the corresponding second position information and projecting each frame of the second image data onto a second three-dimensional volume based on the corresponding second position information; and
generating the image from the first three-dimensional volume or the second three-dimensional model.

20. The method of claim 16, further comprising displaying the image of the subject on a display device.
